(19) United States
(12) Patent Application Publication
(10) Pub. No.: US 2021/0015884 A1
(43) Pub. Date: Jan. 21, 2021

(54) MODIFIED OLIGONUCLEOTIDES AND METHODS OF USE

(71) Applicant: Janssen BioPharma, Inc., South San Francisco, CA (US)

(72) Inventors: Christian Andreas JEKLE, San Francisco, CA (US); Ali R. Ozes, San Bruno, CA (US)

(21) Appl. No.: 17/040,029

(22) PCT Filed: Mar. 21, 2019
(86) PCT No.: PCT/US2019/023463

§ 371 Date: Sep. 21, 2020

Related U.S. Application Data
(60) Provisional application No. 62/646,901, filed on Mar. 22, 2018.

(51) Int. Cl. A61K 38/00; C07H 21/02; A61K 31/7088

(52) U.S. Cl. CPC A61K 38/005; A61K 31/7088; C07H 21/02

(57) ABSTRACT

Methods and compositions for reducing drug resistance are described. Agents for decreasing m⁶A RNA methylation are described. Also described are compositions comprising the agents, methods of making the agents, and methods of using the agents to reduce drug resistance in a subject in need thereof, or to stimulate an immune response.

### FIELD OF THE INVENTION

The disclosure relates to agents, including oligonucleotides, such as 2′-fluoro-adenosine modified RNA oligonucleotides, and methods of reducing drug resistance in a subject treated with a drug. The invention further relates to methods of reducing drug resistance in a subject treated with an anti-viral active agent, and to methods of treating conditions that involve a type I immune response by stimulating an immune response. Conjugates or compositions comprising the oligonucleotides, methods of making the oligonucleotides, and methods of using the oligonucleotides, conjugates or compositions thereof to reduce drug resistance in a subject, or to treat conditions that involve a type I immune response, are also described.

### REFERENCE TO SEQUENCE LISTING SUBMITTED ELECTRONICALLY

This application contains a sequence listing, which is submitted electronically via EFS-Web as an ASCII formatted sequence listing with a file name “ALP0057WOPCT1 Sequence Listing”, creation date of Mar. 21, 2019, and having a size of 4.0 KB. The sequence listing submitted via EFS-Web is part of the specification and is herein incorporated by reference in its entirety.

### BACKGROUND

Drug resistance can be a serious problem, especially resistance against drugs used to treat viral infection. A major problem in the treatment of viral infections is the rapid appearance of drug resistant variants after infection. Influenza is an RNA virus that can cause severe respiratory illness, inflammation that leads to organ failure, and ultimately death. Influenza and other RNA viruses have high mutation rates since there is no repair mechanism for errors in RNA, and seasonal influenza viruses arise as a result of a combination of antigenic shift, which involves mutations introduced by the viral polymerase, and antigenic drift, which involves antigenic variants generated within the viral hemagglutinin (HA) and neuraminidase (NA) proteins that escape existing antibody-mediated immune responses in humans (Iwasaki et al., Nature Reviews Immunology, 2014).

Therapeutic targets of influenza virus include components of the polymerase complex, i.e. PA, PB2 and PB1; the M2 channel; and neuraminidase NA. Neuraminidase inhibitors are the major class of anti-influenza pharmaceuticals. Examples of drugs that target NA include Oseltamavir and Zanamivir. Zanamivir works by binding to the active site of the NA protein, rendering the influenza virus unable to escape its host cell, and Oseltamavir is a competitive inhibitor of NA. While Oseltamavir and Zanamivir are effective therapies in treating influenza, drug resistance can occur in patients being treated with either of the drugs (Colman, Annu. Rev. Biochem., 2009). A pandemic caused by a neuraminidase inhibitor-resistant influenza virus is a serious threat, as the first line defense in pandemic preparedness would be disarmed (Järhult, Acta Vet Scand. 2018 60(1):6).

N6-adenosine methylation (m⁶A) is a modification affecting RNA structure and function (Desrosiers et al., Proc Natl Acad Sci USA, 1974) that is thought to have an effect on drug resistance. The m⁶A modification of RNA is dynamic and reversible. In mammalian cells, m⁶A modification is catalyzed by a complex consisting of methyltransferase-like protein 3 (METTL3), METTL14, and Wilms Tumor 1 Associated Protein (WTAP), and the m⁶A modification is reversed by the demethylase Fat mass and Obesity-associated protein (FTO) and by AlkB Homolog 5 (ALKBH5) (Brocard et al., Journal of General Virology, 2017). Thus, the level of m⁶A is controlled by the host methyltransferase METTL3 and the host demethylase FTO.

Methylated RNAs, such as m⁶A methylated RNAs, are significantly less immunogenic than unmethylated RNAs (McGuinness and McGuinness, Journal of Cancer Science and Clinical Oncology, 2014). In particular, methylation of RNAs, such as m⁶A methylated RNAs, prevents activation of Toll-Like Receptors (TLRs) that play a key role in the innate immune response. Activation of TLRs by unmethylated pathogens, such as bacterial, fungal, parasitic and viral RNA leads to a series of signaling events resulting in the production of type I interferons (IFNs), inflammatory cytokines, and chemokines, and the induction of immune responses (Lichinchi et al., Cell Host and Microbiome, 2016; Narayan et al., Molecular and Cellular Biology, 1987; Kennedy et al., Journal of Virology, 2017). Eventually, this inflammation also activates the adaptive immune system, which then results in the clearance of the invading pathogens and the infected cells. However, pathogenic RNA may become methylated, and methylated viral RNA, for example, evades detection by host TLRs. In fact, increased levels of m⁶A in viral RNA correlates to acquired resistance to treatment of influenza A (IVA) virus by drugs such as Zanamivir. Furthermore, treatment of IVA with 3-Deazaadenosine (3DZA), a chemical inhibitor of m⁶A, decreases drug resistance (Scholtissek and Müller, Archives of Virology, 1991), whereas promotion of m⁶A with meclofenamic acid (MA) increases drug resistance.

Thus, there remains a need for therapeutics that can effectively reduce drug resistance in a subject treated with a drug.

### BRIEF SUMMARY

The present disclosure satisfies the need for therapeutics that can effectively reduce the occurrence of drug resistance in a subject treated with a drug, such as an anti-viral active agent. To that end, the present disclosure provides agents, such as RNA oligonucleotide molecules, that decrease the level of N6-methyladenosine (m⁶A) RNA methylation. Preferably, oligonucleotides of the present disclosure comprise RNA oligonucleotides modified for increased stability.

It has been surprisingly found that oligonucleotide inhibitors according to embodiments of the present disclosure that bind to and serve as steric inhibitors of METTL3 can reduce m⁶A methylation in both host and viral RNA. It has further been found that co-treatment of viral infections such as IVA with the inhibitory oligonucleotides and an antiviral drug such as Zanamivir can inhibit m⁶A levels in viral RNA and result in a reduction of drug-resistant virus.

In a general aspect, the disclosure relates to a method of reducing drug resistance in a subject in need thereof, such as a subject in need of a treatment with an anti-viral active agent, the method comprising administering to the subject an effective amount of an agent, such as a disclosed oligonucleotide, that decreases the level of m⁶A RNA methylation in the subject, thereby reducing drug resistance in the subject.

In an embodiment of the application, the method comprises administering to the subject an inhibitor of at least one of METTL3, METTL14, and WTAP.

In an embodiment of the application, the method comprises administering to the subject an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6.

According to a particular aspect, one or more nucleosides in the disclosed oligonucleotide is modified.

According to a particular aspect, the disclosed oligonucleotide consists of the polynucleotide sequence of 5′ GGACUGGACUGGACUGGACU 3′ (SEQ ID NO: 1), and optionally, one or more nucleoside in the oligonucleotide is modified.

According to a particular aspect, the disclosed oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

According to a particular aspect, the subject is administered with an effective amount of a disclosed oligonucleotide that decreases the level of m⁶A RNA methylation in combination with an anti-influenza drug, preferably a neuraminidase inhibitor, such as Zanamivir or Oseltamivir.

In another general aspect, the disclosure relates to a method of stimulating an immune response in a subject in need thereof, the method comprising administering to the subject an effective amount of a disclosed oligonucleotide that decreases the level of m⁶A RNA methylation in the subject, thereby stimulating an immune response in the subject.

In a general aspect, the disclosure relates to an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

In a general aspect, the disclosure relates to a pharmaceutical composition comprising an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine, and a pharmaceutically acceptable carrier.

According to a particular aspect, the pharmaceutical composition further comprises an anti-viral active agent, preferably an anti-influenza drug, more preferably a neuraminidase inhibitor, such as Zanamivir or Oseltamivir.

In a general aspect, the disclosure relates to a kit comprising an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine, and an anti-viral active agent, wherein the oligonucleotide and the anti-viral active agent are present in the same composition or different compositions, and wherein the anti-viral active agent is an anti-influenza drug, preferably a neuraminidase inhibitor, such as Zanamivir or Oseltamivir.

Other aspects, features and advantages of the disclosed embodiments will be apparent from the following disclosure, including the detailed description and its preferred embodiments and the appended claims.

### BRIEF DESCRIPTION OF THE DRAWINGS

The foregoing summary, as well as the following detailed description, will be better understood when read in conjunction with the appended drawings. It should be understood that the invention is not limited to the precise embodiments shown in the drawings.

In the drawings:

FIG. 1A shows the doses of Zanamavir used during resistance passaging;

FIG. 1B shows EC50 values of Zanamivir-passaged Influenza A/PC;

FIG. 2 shows the fold change in m⁶A levels in Influenza A/PC and Influenza B/Vic viral RNA before and after resistance passaging with Zanamavir (ZanR);

FIG. 3 shows the m⁶A levels in control samples and in samples treated with 3DZA or MA;

FIG. 4 shows the m⁶A levels in Influenza A/PC or Influenza A/PC/ZanR strains treated with 3DZA or MA;

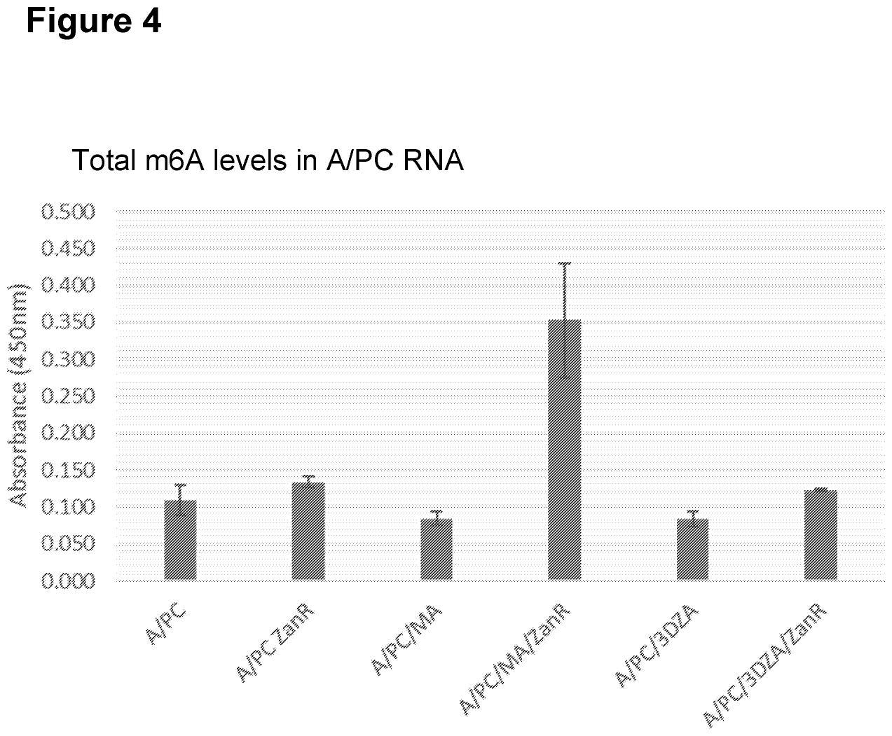

FIG. 5 shows the EC50 values of Influenza A/PC or Influenza A/PC/ZanR strains treated with 3DZA or MA;

FIG. 6 shows a schematic (left) and the results (right) of an immunoprecipitation assay of biotin-labeled oligonucleotides;

FIG. 7A shows m⁶A levels in MDCK cells after treatment with disclosed RNA oligonucleotides;

FIG. 7B shows m⁶A levels in HEK293 cells after treatment with disclosed RNA oligonucleotides of the present disclosure;

FIG. 8 shows the m⁶A levels in MDCK cells infected with Influenza A/PC or Influenza A/PC/ZanR strains after treatment with disclosed RNA oligonucleotides of the present disclosure; and

FIG. 9 shows the EC50 values of Influenza A/PC or Influenza A/PC/ZanR strains after treatment with disclosed RNA oligonucleotides of the present disclosure.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

### Definitions

Unless defined otherwise, all technical and scientific terms used herein have the same meaning commonly understood to one of ordinary skill in the art to which the present disclosure pertains. Otherwise, certain terms used herein have the meanings as set in the specification. All patents, published patent applications and publications cited herein are incorporated by reference as if set forth fully herein. It must be noted that as used herein and in the appended claims, the singular forms “a,” “an,” and “the” include plural reference unless the context clearly dictates otherwise.

As used herein, the terms “decrease”, “reduce” or “inhibit” all refer to a decrease by a statistically significant amount. According to particular embodiments of the present disclosure, “decrease”, “reduce” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease, or any decrease between 10 and 100% as compared to a reference level.

As used herein, the term “statistically significant” or “significantly” refers to statistical significance and generally means a two standard deviation or greater difference in a value of the measurement. The term refers to statistical evidence that there is a difference and is defined as the probability of making a decision to reject the null hypothesis when the null hypothesis is actually true. Statistical significance can be determined, e.g., by t-test or using a p-value.

As used herein, the term “a subject treated with a drug” or “a subject treated with an active agent” refers to a subject that is treated with a drug or an active agent before, during, or after administration of an agent, such as a disclosed oligonucleotide, that decreases the level of m⁶A RNA methylation according to embodiments of the present disclosure. According to particular embodiments of the present disclosure, the agent that decreases the level of m⁶A RNA methylation, such as an RNA oligonucleotide, is administered to a subject who is currently being treated with a drug or active agent. According to other particular embodiments of the present disclosure, the disclosed agent that decreases the level of m⁶A RNA methylation, such as an RNA oligonucleotide, is administered prophylactically to subjects who will undergo treatment with a drug or active agent in the future. According to other particular embodiments of the present disclosure, the agent that decreases the level of m⁶A RNA methylation, such as an RNA oligonucleotide, is administered to a subject who has previously undergone treatment with a drug or active agent.

As used herein, the term “anti-viral active agent” refers to any compound that is used to treat or prevent a viral infection in a subject. According to particular embodiments of the present disclosure, the anti-viral active agent is a compound that is used to treat or prevent infection by any virus, the drug resistance of which is regulated by m⁶A RNA methylation, such as double-stranded (ds)/single-stranded (ss) RNA or DNA viruses, such as influenza virus, HIV, or Zika virus.

As used herein, the term “effective amount” refers to an amount of an active ingredient or component that elicits the desired biological or medicinal response in a subject. An effective amount can be determined empirically and/or in a routine manner, in relation to the stated purpose. For example, in vitro assays can optionally be employed to help identify optimal dosage ranges. Selection of a particular effective dose can be determined (e.g., via clinical trials) by those skilled in the art based upon the consideration of several factors, including the disease to be treated or prevented, the symptoms involved, the patient's body mass, the patient's immune status and other factors known by the skilled artisan. The precise dose to be employed in the formulation will also depend on the route of administration and the severity of disease, and should be decided according to the judgment of the practitioner and each patient's circumstances. Effective doses can be extrapolated from dose-response curves derived from in vitro or animal model test systems.

As used herein, the term “N6-methyladenosine”, “m⁶A” or “m6A” refers to methylation of the adenosine base at the nitrogen-6 position of RNA in a cell.

As used herein, the term “subject” refers to an animal. According to particular embodiments, the subject is a mammal including a non-primate (e.g., a camel, donkey, zebra, cow, pig, horse, goat, sheep, cat, dog, rat, rabbit, guinea pig or mouse) or a primate (e.g., a monkey, chimpanzee, or human). In particular embodiments, the subject is a human.

As used herein, the term “inhibitor” refers to a compound or molecule that prevents or decreases the amount or the activity of a protein. For example, the term “inhibitor” can refer to a compound or molecule that negatively regulates the expression, stability or activity of a protein, including, but not limited to, transcription of a protein mRNA, stability of a protein mRNA, translation of a protein mRNA, stability of a protein polypeptide, a protein post-translational modification, a protein activity, a protein signaling pathway or any combination thereof. Inhibitors of METTL3, METTL14 or WTAP include, but are not limited to, nucleic acids, such as nucleic acid inhibitors that bind to and inhibit one or more of the proteins, or antisense nucleic acids that reduce or prevent expression of one or more of the proteins; small molecule inhibitors that inhibit activity of one or more of the proteins; peptides or proteins that bind to and inhibit activity of one or more of the proteins, such as antibodies that selectively bind to one or more of the proteins and inhibit its activity; carbohydrates, lipids or any other molecules that reduce the level or activity of one or more of the proteins. According to particular aspects, inhibitors include, e.g., oligonucleotides that inhibit (sterically or otherwise) one or more of the proteins, siRNA molecules that target the transcript of one or more of the proteins, or antibodies or small molecules that inhibit the activity of one or more of the proteins.

As used herein, the term “Methyltransferase-Like Protein 3” or “METTL3”, also known as N6-adenosine-methyltransferase 70 kDa subunit or MT-A70, refers to a component of the METTL3-METTL14 heterodimer that forms a N6-methyltransferase complex that methylates adenosine residues at the N6 position of some RNAs and regulates various processes such as the circadian clock, differentiation of embryonic and hematopoietic stem cells, cortical neurogenesis, response to DNA damage, differentiation of T-cells and primary miRNA processing. Examples of METTL3 include, but are not limited to, a human METTL3 that is a 580 amino acid-long protein encoded by an mRNA transcript 2038 nucleotides long (NM_019852.4). The amino acid sequence of the exemplified human METTL3 is represented in GenBank Accession No. NP_062826.2. As used herein, the term “METTL3” includes homologs of METTL3 from species other than human, such as Macaca Fascicularis (cynomolgous monkey) or Pan troglodytes (chimpanzee). As used herein, the term “METTL3” includes proteins comprising mutations, e.g., point mutations, fragments, insertions, deletions and splice variants of full length wild type METTL3. The term “METTL3” also encompasses post-translational modifications of the METTL3 amino acid sequence.

As used herein, the term “Methyltransferase-Like Protein 14” or “METTL14”, also known as KIAA1627, refers to a component of the METTL3-METTL14 heterodimer described above. Examples of METTL14 include, but are not limited to, a human METTL14 that is a 456 amino acid-long protein encoded by an mRNA transcript 3520 nucleotides long (NM_020961.3). The amino acid sequence of the exemplified human METTL14 is represented in GenBank Accession No. NP_066012.1. As used herein, the term “METTL14” includes homologs of METTL14 from species other than human, such as Macaca Fascicularis (cynomolgous monkey) or Pan troglodytes (chimpanzee). As used herein, the term “METTL14” includes proteins comprising mutations, e.g., point mutations, fragments, insertions, deletions and splice variants of full length wild type METTL14. The term “METTL14” also encompasses post-translational modifications of the METTL14 amino acid sequence.

As used herein, the term “Wilms Tumor 1 Associated Protein” or “WTAP”, also known as KIAA1627, refers to the regulatory subunit of the METTL3-METTL14 N6-methyltransferase complex described above. Examples of WTAP include, but are not limited to, a human WTAP that is a 396 amino acid-long protein encoded by an mRNA transcript 2265 nucleotides long (NM_004906.4). The amino acid sequence of the exemplified human WTAP is represented in GenBank Accession No. NP_004897.2. As used herein, the term “WTAP” includes homologs of WTAP species other than human, such as Macaca Fascicularis (cynomolgous monkey) or Pan troglodytes (chimpanzee). As used herein, the term “WTAP” includes proteins comprising mutations, e.g., point mutations, fragments, insertions, deletions and splice variants of full length wild type WTAP. The term “WTAP” also encompasses post-translational modifications of the WTAP amino acid sequence.

As used herein, the term “oligonucleotide” refers to a polynucleotide formed from a plurality of linked nucleotide units (i.e., ribonucleotides, deoxyribonucleotides, or both). Such oligonucleotides can be obtained from existing nucleic acid sources or can be produced by synthetic methods. In some embodiments, the oligonucleotides each have from about 10 to about 30 nucleotide residues, preferably from about 10 to about 25 nucleotide residues, more preferably about 10 to about 20 nucleotide residues. According to particular embodiments, internucleotide linkages for the oligonucleotides include, but are not limited to, phosphodiester linkages, phosphothioate linkages, and mixtures thereof.

As used herein, the term “in combination,” in the context of the administration of two or more therapies to a subject, refers to the use of more than one therapy. The use of the term “in combination” does not restrict the order in which therapies are administered to a subject. For example, a first therapy (e.g., a composition described herein) can be administered prior to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 16 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy to a subject. Alternatively, for example, a first therapy and a second therapy can be administered simultaneously, either in the same composition or in separate compositions.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an RNA oligonucleotide-based pharmaceutical composition can be used in the invention.

As used herein, the terms “induce” and “stimulate” and variations thereof refer to any measurable increase in cellular activity. Induction of an immune response can include increasing the proliferation of B cells, producing antigen-specific antibodies, increasing the proliferation of antigen-specific T cells, improving dendritic cell antigen presentation and/or an increasing expression of certain cytokines, chemokines and co-stimulatory markers.

As used herein, the terms “treat,” “treating,” and “treatment” are all intended to refer to an amelioration or reversal of at least one measurable physical parameter related to a disease in which stimulation of an immune response would be beneficial, such as an immune disease, disorder or condition, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease in which stimulation of an immune response would be beneficial such as an immune disease, disorder or condition, including a viral infection or a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

As used herein a “disease in which stimulation of an immune response would be beneficial” include any disease in which stimulation of an immune response would benefit the subject. For example, a disease in which stimulation of an immune response would be beneficial can include immune diseases, disorders or conditions. According to particular embodiments, the disease, disorder or condition to be treated is an inflammatory disease, disorder or condition, an autoimmune disease, disorder or condition, or a disease, disorder or condition caused by a pathogen, such as a viral infection. According to particular embodiments, the disease in which stimulation of an immune response would be beneficial is a viral infection that is an RNA or DNA virus such as adenovirus, cytomegalovirus, hepatitis A virus (HAV), hepadnaviruses including HBV, chronic HBV, flaviviruses including Yellow Fever virus, hepaciviruses including hepatitis C virus (HCV), herpes simplex type 1 and 2, herpes zoster, human herpesvirus 6, human immunodeficiency virus (HIV), Zika virus, human papilloma virus (HPV), influenza A virus, influenza B virus, measles, parainfluenza virus, pestivirus, poliovirus, poxvirus, rhinovirus, coronovirus, respiratory syncytial virus (RSV), multiple families of viruses that cause hemorrhagic fevers, including the Arenaviruses, the Bunyaviruses and Filoviruses, and a range of viral encephalitides caused by RNA and DNA viruses.

### Methods of Reducing Drug Resistance or Stimulating an Immune Response

In a general aspect, the disclosure relates to a method of reducing drug resistance in a subject treated with a drug such as an anti-viral agent, the method comprising administering to the subject an effective amount of an agent, such as a disclosed oligonucleotide. In an embodiment, the agent, such as the disclosed oligonucleotide, decreases the level of m⁶A RNA methylation in the subject, thereby reducing drug resistance in the subject.

The present disclosure is also directed to a method of reducing drug resistance in a subject treated with a drug, the method comprising administering to the subject an effective amount of an agent, such as a nucleic acid inhibitor, that inhibits at least one of METTL3, METTL14, and WTAP, thereby decreasing the level of N6-methyladenosine (m⁶A) RNA methylation in the subject.

The present disclosure is further directed to a method of reducing drug resistance in a subject treated with a drug, the method comprising administering to the subject an effective amount of an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6, optionally one or more nucleoside in the oligonucleotide is modified.

In an embodiment, the polynucleotide sequence of the oligonucleotide is (GGACU)n, where n is 2-6.

In an embodiment, the polynucleotide sequence of the oligonucleotide is (GG/i2FA/CU)n, where i2FA represents 2′-fluoro-adenosine and n is 2-6.

The presence or level of drug resistance in a subject caused by administration of that drug to the subject, and the effect of an agent, such as the disclosed nucleic acid inhibitor, on the presence or level of drug resistance, can be determined using methods known in the art in view of the present disclosure. Exemplary methods are described herein, e.g., in the Examples below.

According to particular embodiments of the present disclosure, the anti-viral active agent is an anti-influenza drug (e.g., a neuraminidase inhibitor, e.g., Zanamivir, Oseltamivir, Tamiphosphor, Peramivir), an anti-HIV drug (e.g., AZT, ddC, TiBO derivatives, acyclovir, alpha-interferon), an anti-Zika drug, or an immunostimulant or immunomodulator (e.g., an interleukin, a cytokine). According to particular embodiments of the present disclosure, the anti-viral active agent is an anti-influenza drug selected from Zanamivir and Oseltamivir.

In another general aspect, the disclosure relates to a method of stimulating an immune response in a subject in need thereof, the method comprising administering to the subject an effective amount of an agent, such as a disclosed nucleic acid inhibitor. In an embodiment, the disclosed nucleic acid inhibitor decreases the level of m⁶A RNA methylation in the subject, thereby stimulating an immune response in the subject.

Stimulation of an immune response can be determined using methods known in the art in view of the present disclosure. Exemplary methods include, e.g., measuring an immune response using ELISAs or antibodies specific to cytokines and chemokines, measuring a response of immune cells (e.g., Peripheral Blood Mononuclear Cells (PBMCs), consisting of lymphocytes and monocytes) or reporter cells contacted with an agent, such as the disclosed nucleic acid inhibitor, or measuring cytokine induction in an animal after injection with the disclosed nucleic acid inhibitor.

The level of m⁶A RNA methylation in the subject, and the effect of an agent, such as disclosed nucleic acid inhibitor, on the level of m⁶A RNA methylation in the subject, can be determined using methods known in the art in view of the present disclosure. For example, m⁶A can be measured using ELISAs or antibodies that bind to and can isolate m⁶A-modified mRNAs to enable their quantification. Exemplary methods are described herein, e.g., in the Examples below.

As used herein with reference to an agent, such as a disclosed nucleic acid inhibitor, that decreases the level of m⁶A RNA methylation, an effective amount means an amount of the agent, such as the disclosed nucleic acid inhibitor, that reduces the level of m⁶A RNA methylation in a subject in need thereof, and thereby reduces anti-viral drug resistance of the virus for which the subject is treated with an anti-viral active agent or induces an immune response in a subject in need thereof. Also as used herein with reference to an agent, such as a disclosed nucleic acid inhibitor, that decreases the level of m⁶A RNA methylation, an effective amount means an amount of the agent, such as the disclosed nucleic acid inhibitor, that results in treatment of the viral infection for which the subject is treated with an anti-viral active agent; prevents or slows the progression of the viral infection for which the subject is treated with an anti-viral active agent; reduces or completely alleviates symptoms associated with the viral infection for which the subject is treated with an anti-viral active agent. Also as used herein with reference to an agent, such as a disclosed nucleic acid inhibitor, that decreases the level of m⁶A RNA methylation, an effective amount means an amount of the agent, such as the disclosed nucleic acid inhibitor that results in treatment of the a disease, disorder or condition in which stimulation of an immune response would be beneficial; prevents or slows the progression of the disease, disorder or condition in which stimulation of an immune response would be beneficial; reduces or completely alleviates symptoms associated with the disease, disorder or condition in which stimulation of an immune response would be beneficial.

According to particular embodiments, an effective amount refers to the amount of therapy which is sufficient to achieve one, two, three, four, or more of the following effects: (i) reduce or ameliorate the severity of the viral infection for which the subject is treated with an anti-viral active agent, or a symptom associated therewith; (ii) reduce the duration of the viral infection for which the subject is treated with an anti-viral active agent, or a symptom associated therewith; (iii) prevent the progression of the viral infection for which the subject is treated with an anti-viral active agent, or a symptom associated therewith; (iv) cause regression of the viral infection for which the subject is treated with an anti-viral active agent, or a symptom associated therewith; (v) prevent the development or onset of the viral infection for which the subject is treated with an anti-viral active agent, or a symptom associated therewith; (vi) prevent the recurrence of the viral infection for which the subject is treated with an anti-viral active agent, or a symptom associated therewith; (vii) reduce hospitalization of a subject having the viral infection for which the subject is treated with an anti-viral active agent, or a symptom associated therewith; (viii) reduce hospitalization length of a subject having the viral infection for which the subject is treated with an anti-viral active agent, or a symptom associated therewith; (ix) increase the survival of a subject with the viral infection for which the subject is treated with an anti-viral active agent, or a symptom associated therewith; (xi) inhibit or reduce the viral infection for which the subject is treated with an anti-viral active agent, or a symptom associated therewith in a subject; and/or (xii) enhance or improve the prophylactic or therapeutic effect(s) of another therapy.

According to other particular embodiments, an effective amount refers to the amount of therapy which is sufficient to achieve one, two, three, four, or more of the following effects: (i) reduce or ameliorate the severity of the disease, disorder or condition in which stimulation of an immune response would be beneficial, or a symptom associated therewith; (ii) reduce the duration of the disease, disorder or condition in which stimulation of an immune response would be beneficial, or a symptom associated therewith; (iii) prevent the progression of the disease, disorder or condition in which stimulation of an immune response would be beneficial, or a symptom associated therewith; (iv) cause regression of the disease, disorder or condition in which stimulation of an immune response would be beneficial, or a symptom associated therewith; (v) prevent the development or onset of the disease, disorder or condition in which stimulation of an immune response would be beneficial, or a symptom associated therewith; (vi) prevent the recurrence of the disease, disorder or condition in which stimulation of an immune response would be beneficial, or a symptom associated therewith; (vii) reduce hospitalization of a subject having the disease, disorder or condition in which stimulation of an immune response would be beneficial, or a symptom associated therewith; (viii) reduce hospitalization length of a subject having the disease, disorder or condition in which stimulation of an immune response would be beneficial, or a symptom associated therewith; (ix) increase the survival of a subject with the disease, disorder or condition in which stimulation of an immune response would be beneficial, or a symptom associated therewith; (xi) inhibit or reduce the disease, disorder or condition in which stimulation of an immune response would be beneficial, or a symptom associated therewith in a subject; and/or (xii) enhance or improve the prophylactic or therapeutic effect(s) of another therapy.

The therapeutically effective amount or dosage can vary according to various factors, such as the means of administration, the physiological state of the subject (including, e.g., age, body weight, health), whether the subject is a human or an animal, and other medications administered. Treatment dosages are optimally titrated to optimize safety and efficacy.

The mode of administration for therapeutic use of an agent, such as a nucleic acid inhibitor, of the present disclosure can be any suitable route that delivers the agent, such as the disclosed nucleic acid inhibitor, to the host. For example, the compositions described herein can be formulated to be suitable for parenteral administration, e.g., intradermal, intramuscular, intraperitoneal, intravenous, subcutaneous, intranasal or intracranial administration, or they can be administered into the cerebrospinal fluid of the brain or spine.

According to particular embodiments, the viral infection for which the subject is treated with an anti-viral active agent is an infection by any virus for which the drug resistance is regulated by m⁶A RNA methylation. For example, the viral infection can include an influenza virus, HIV, Ebola, HCV or Zika virus. According to particular embodiments, the viral infection is an influenza virus infection, such as an influenza A virus infection, an influenza B virus infection, or an influenza C virus infection. According to particular embodiments, the viral infection is an influenza A virus infection.

According to particular embodiments, the agent is an inhibitor of at least one of METTL3, METTL14, and WTAP. According to particular embodiments, the inhibitor is a nucleic acid, such as an oligonucleotide that binds to one or more of METTL3, METTL14, or WTAP gene, or an antisense nucleic acid that reduces or prevents expression of METTL3, METTL14, or WTAP. According to particular embodiments, the inhibitor is a small molecule, peptide, antibody, carbohydrate or lipid that binds to and inhibits the activity of METTL3, METTL14, or WTAP. According to particular embodiments, the inhibitor is a nucleic acid inhibitor that blocks or decreases the binding between one or more of the proteins and the consensus sequence of the N6-methyltransferase complex, RRm⁶ACH, wherein R is independently guanosine or adenosine, C is cytidine, and H is independently adenosine, cytidine or uridine.

According to particular aspects, the nucleic acid inhibitor is an RNA oligonucleotide that binds to and sterically inhibits METTL3. According to particular embodiments, the nucleic acid inhibitor is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, n is an integer of 2 to 6, and one or more nucleoside in the oligonucleotide is modified. According to particular embodiments, the (RRACH) moieties are adjacent to one another, and do not have other nucleotides between them. According to particular embodiments, the disclosed nucleic acid inhibitor is an RNA oligonucleotide comprising one or more of GGACA, GGACC, GGACU, GAACA, GAACC, GAACU, AGACA, AGACC, AGACU, GGm⁶ACA, GGm⁶ACC, GGm⁶ACU, GAm⁶ACA, GAm⁶ACC, GAm⁶ACU, AGm⁶ACA, AGm⁶ACC and AGm⁶ACU, or a combination thereof, such as GGACAGGACCGGACU GAACA, GAACCGAACU AGACAAGACC, (GGACA)₂(AGACU)₂, GAACU(AGACA)₃AGACC, etc. According to particular embodiments, the RNA oligonucleotide consists of the polynucleotide sequence of 5′ GGACUGGACUGGACUGGACU 3′ (SEQ ID NO: 1), and optionally, one or more nucleoside in the RNA oligonucleotide is modified.

Preferably one or more nucleoside in the oligonucleotide is modified to increase the stability of the oligonucleotide. According to particular aspects, the RNA oligonucleotide is stabilized by one or more modifications using chemistry known in the art. Exemplary nucleotide modifications include sugar- and/or phosphate backbone-modified ribonucleotides. For example, the phosphodiester linkages of natural RNA can be modified to include at least one of a nitrogen or sulfur heteroatom. In exemplary backbone-modified ribonucleotides, at least one of the phosphodiester groups connecting to adjacent ribonucleotides is replaced by a modified group, such as a phosphorothioate group, phosphoramidate or thiophosphoramidate. In exemplary sugar-modified ribonucleotides, the 2′ OH-group of the sugar is replaced by a group selected from H, R, OR, OROR, halo, SH, SR, NH₂, NHR, NR₂ or ON, wherein each R is independently C₁-C₂ alkyl, haloalkyl, alkenyl or alkynyl and halo is F, Cl, Br or I. In particular embodiments, the 2′ OH-group of the sugar is replaced by fluoro. In particular embodiments, the 2′ OH-group of the sugar is replaced by fluoro—a 2′ fluoro modification. In particular embodiments, the 2′ OH-group of the sugar is replaced by fluoro in a nucleoside having an adenine base—a 2′-fluoro-adenosine modification.

In embodiments, one or more adenosine in the oligonucleotide of the present disclosure is modified with a 2′-fluoro, 2′-O-methyl, 2′-O-methoxy, 2′-O-ethyl, or 2′-O-methoxyethyl. The 2′-sugar substituent groups can be in the arabino (up) position or ribo (down) position.

Other modifications can include, but are not limited to, 2′-amino and/or 2′-thio modifications. Particular modifications include 2′-fluoro-cytidine, 2′-fluoro-uridine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine.

The sugar-modified ribonucleotides can also be modified at other positions of the sugar, such as the 5′-position. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. Additional modified residues include, inosine, N3-methyl-uridine, N6,N6-dimethyl-adenosine, pseudouridine, purine ribonucleoside and ribavirin. It should be appreciated that more than one chemical modification can be combined within the same molecule. It should also be appreciated that any one or more nucleosides in the RNA oligonucleotide can be modified.

According to particular aspects, one or more nucleoside in the RNA oligonucleotide is modified by a 2′-fluoro-adenosine modification. In an embodiment, the polynucleotide sequence is (GG/i2FA/CU)n, where i2FA represents 2′-fluoro-adenosine and n is 2-6. According to particular aspects, the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

According to particular embodiments, an oligonucleotide of the present disclosure can be conjugated via a linker to a targeting moiety. According to particular embodiments, the targeting moiety increases the stability and/or directs the delivery of the conjugated oligonucleotide.

As used herein, the term “linker” refers to a chemical moiety that joins a nucleotide to a targeting moiety, and the term “cleavable linker” refers to a linker that can be cleaved to remove the potentiating moiety from the nucleotide when desired, essentially without altering the nucleotide or the nucleic acid molecule to which it is attached. Cleavage can be accomplished, for example, by acid or base treatment, by oxidation or reduction of the linkage, by light treatment (photobleaching), depending upon the nature of the linkage. The linkers can be, for example, a single covalent bond, a substituted or unsubstituted alkyl, a substituted or unsubstituted heteroalkyl moieties, a polyethylene glycol (PEG) linker or one, two, or three abasic and/or ribitol groups.

As used herein, the term “targeting moiety” refers to any moiety suitable for stabilizing and/or directing the delivery of its conjugated oligonucleotide to targeted cells. According to particular embodiments, the targeting moiety is a lipid moiety. As used herein, a “lipid moiety” refers to a moiety containing a lipophilic structure. Lipid moieties, such as cholesterol, tocopherol or other fatty acids, when attached to highly hydrophilic molecules, such as nucleic acids, can substantially enhance plasma protein binding and consequently circulation half-life of the hydrophilic molecules.

According to particular embodiments, the targeting moiety binds to receptors present on the particular target cell types of interest. The targeting moiety helps in targeting the oligonucleotide to the required target site. One way a targeting moiety can improve delivery is by receptor-mediated endocytotic activity. This mechanism of uptake involves the movement of the oligonucleotide bound to membrane receptors into the interior of an area that is enveloped by the membrane via invagination of the membrane structure or by fusion of the delivery system with the cell membrane, and it is initiated via activation of a cell-surface or membrane receptor following binding of a specific ligand to the receptor. Many receptor-mediated endocytotic systems are known and have been studied, including those that recognize sugars such as galactose, mannose, mannose-6-phosphate, peptides and proteins such as transferrin, asialoglycoprotein, vitamin B12, insulin and epidermal growth factor (EGF).

According to particular aspects, the subject is administered with the effective amount of the disclosed nucleic acid inhibitor in combination with an anti-viral active agent, such as an anti-influenza drug, an anti-HIV drug or an anti-Zika virus drug. According to particular aspects, the subject is administered with the effective amount of the disclosed nucleic acid inhibitor in combination with an anti-influenza drug, such as Zanamivir or Oseltamivir. According to other particular aspects, the subject is administered with the effective amount of the disclosed nucleic acid inhibitor in combination with an immune modulator or an anti-inflammatory drug. According to particular aspects, the subject is administered with the effective amount of the disclosed nucleic acid inhibitor in combination with an immune modulator, such as an agonist for TLR7 or TLR9.

The disclosed nucleic acid inhibitor can be administered in a single dose schedule, or as a multiple dose schedule in which a primary course of treatment including 1-10 separate doses is followed by other doses given at subsequent time intervals, for example, at 1-4 days, weeks or months for a second dose, and if needed, a subsequent dose(s) after several days, weeks or months.

### Oligonucleotides, Pharmaceutical Compositions, and Kits

In an embodiment, the polynucleotide sequence of (RRACH)n is (GG/i2FA/CU)n, where i2FA represents 2′-fluoro-adenosine and n is 2-6. In embodiments, the nucleic acid inhibitor is an oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

The disclosed nucleic acid inhibitors such as RNA oligonucleotides can be made using methods known in the art in view of the present disclosure. For example, the disclosed RNA oligonucleotides can be made with solid phase synthesis, see for example “Oligonucleotide synthesis, a practical approach”, Ed. M. J. Gait, IRL Press, 1984; “Oligonucleotides and Analogues, A Practical Approach”, Ed. F. Eckstein, IRL Press, 1991 (e.g. Chapter 1, Modern machine-aided methods of oligodeoxyribonucleotide synthesis, Chapter 2, Oligoribonucleotide synthesis, Chapter 4, Phosphorothioate oligonucleotides, Chapter 5, Synthesis of oligonucleotide phosphorodithioates). Other particularly useful synthetic procedures, reagents, blocking groups and reaction conditions are described in Martin, P., Helv. Chim. Acta, 1995, 78, 486-504; Beaucage, S. L. and Iyer, R. P., Tetrahedron, 1992, 48, 2223-2311 and Beaucage, S. L. and Iyer, R. P., Tetrahedron, 1993, 49, 6123-6194, or references referred to therein. Certain of the disclosed oligonucleotides and monomers thereof can also be obtained commercially.

In a general aspect, the disclosure relates to a pharmaceutical composition comprising an a nucleic acid inhibitor consisting of 5′ GG/i2FA/CUGG/i2FA/CUGG/i2FA/CUGG/i2FA/CU 3′ (SEQ ID NO: 2), wherein i2FA represents 2′-fluoro-adenosine, and a pharmaceutically acceptable carrier.

According to particular embodiments, the compositions described herein are formulated to be suitable for the intended route of administration to a subject. For example, the compositions described herein can be formulated to be suitable for intravenous, subcutaneous or intramuscular administration. According to preferred embodiments, the compositions described herein are formulated to be suitable for intravenous or subcutaneous administration.

According to another general aspect, the application relates to a pharmaceutical composition comprising an agent, such as a nucleic acid inhibitor, of the present disclosure and another active ingredient, such as an anti-viral active agent. In one embodiment of the application, the pharmaceutical composition comprises an anti-influenza drug, an anti-HIV drug, an anti-Zika virus drug, an immunostimulant or an immunomodulator, in combination with an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6. In an embodiment, one or more nucleosides in the oligonucleotide is modified. According to particular aspects, the anti-influenza drug in the pharmaceutical composition comprises a neuraminidase inhibitor, such as Zanamivir or Oseltamivir. In an embodiment, the RNA oligonucleotide has a polynucleotide sequence of 5′ GGACUGGACUGGACUGGACU 3′ (SEQ ID NO: 1), which optionally has one or more nucleosides modified, and preferably consists of 5′ GG/i2FA/CUGG/i2FA/CUGG/i2FA/CUGG/i2FA/CU 3′ (SEQ ID NO: 2), wherein i2FA represents 2′-fluoro-adenosine.

In another general aspect, the disclosure relates to a method of producing a pharmaceutical composition comprising an agent, such as a disclosed nucleic acid inhibitor, comprising combining the agent or the nucleic acid inhibitor with a pharmaceutically acceptable carrier to obtain the pharmaceutical composition.

In another general aspect, the disclosure relates to a method of treating a disease in a subject in need thereof, comprising administering to the subject the pharmaceutical composition of the present disclosure, wherein the disease is a disease in which stimulation of an immune response would be beneficial. According to particular aspects, the disease is a viral infection or disease, disorder or condition, such as an infection with influenza virus, Ebola virus, Zika virus, HIV, rhinovirus, HCV, and HBV.

In a general aspect, the disclosure relates to a kit comprising an agent, such as a disclosed nucleic acid inhibitor, and another active ingredient, such as an anti-viral active agent, wherein the disclosed nucleic acid inhibitor and the other active ingredient are present in the same composition or different compositions. In an embodiment, the agent or nucleic acid inhibitor is effective in decreasing the level of m⁶A RNA methylation. In one embodiment of the application, the kit comprises an anti-influenza drug, an anti-HIV drug, an anti-Zika virus drug, an immunostimulant or an immunomodulator, and an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, n is an integer of 2 to 6, and one or more nucleoside in the oligonucleotide is modified. According to particular aspects, the kit comprises an anti-influenza drug that is a neuraminidase inhibitor, which is preferably selected from Zanamivir and Oseltamivir, and an RNA oligonucleotide consisting of the polynucleotide sequence of 5′ GGACUGGACUGGACUGGACU 3′ (SEQ ID NO: 1), which optionally has one or more nucleoside modified, and preferably consists of 5′ GG/i2FA/CUGG/i2FA/CUGG/i2FA/CUGG/i2FA/CU 3′ (SEQ ID NO: 2), wherein i2FA represents 2′-fluoro-adenosine.

The contents of all cited references (including literature references, issued patents, published patent applications, and co-pending patent applications) cited throughout this application are hereby expressly incorporated by reference.

### Embodiments

The invention provides also the following non-limiting embodiments.

Embodiment 1 is a method of reducing drug resistance in a subject treated with a drug, the method comprising administering to the subject an effective amount of an agent that decreases the level of N6-methyladenosine (m⁶A) RNA methylation in the subject, thereby reducing drug resistance in the subject.

Embodiment 2 is the method of Embodiment 1, wherein the agent is an inhibitor of at least one of Methyltransferase-Like Protein 3 (METTL3), METTL14, and Wilms Tumor 1 Associated Protein (WTAP).

Embodiment 3 is the method of Embodiment 1 or 2, wherein the agent is a nucleic acid, a small molecule, or polypeptide such as an antibody or an antigen binding fragment thereof.

Embodiment 4 is the method of any of Embodiments 1 to 3, wherein the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6, optionally one or more nucleoside in the oligonucleotide is modified.

Embodiment 5 is the method of Embodiment 4, wherein the polynucleotide sequence is (GGACU)n, wherein n is an integer of 2 to 6.

Embodiment 6 is the method of Embodiment 4 or 5, wherein the oligonucleotide consists of the polynucleotide sequence of 5′ GGACUGGACUGGACUGGACU 3′ (SEQ ID NO: 1), which optionally contains one or more nucleoside modifications.

Embodiment 7 is the method of any one of Embodiments 4 to 6, wherein one or more nucleosides in the oligonucleotide is modified.

Embodiment 8 is the method of Embodiment 7, wherein one or more nucleoside in the oligonucleotide has a 2′-fluoro modification, preferably, one or more adenosine residues in the oligonucleotide has a 2′-fluoro modification.

Embodiment 9 is the method of any of Embodiments 6 to 8, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 10 is the method of any of Embodiments 4 to 9, wherein the oligonucleotide is conjugated via a linker to a targeting moiety.

Embodiment 11 is the method of any of Embodiments 4 to 10, wherein the oligonucleotide is administered intravenously or subcutaneously.

Embodiment 12 is the method of any of Embodiments 1 to 11, wherein the subject is administered with the effective amount of the agent in combination with the drug.

Embodiment 13 is the method of any of Embodiments 1 to 12, wherein the drug is an anti-viral drug.

Embodiment 14 is the method of Embodiment 13, wherein the anti-viral drug is an anti-influenza drug.

Embodiment 15 is the method of Embodiment 14, wherein the anti-influenza drug is a neuraminidase inhibitor.

Embodiment 16 is the method of Embodiment 15, wherein the neuraminidase inhibitor is Zanamivir or Oseltamivir.

Embodiment 17 is a method of stimulating an immune response in a subject in need thereof, the method comprising administering to the subject an effective amount of an agent that decreases the level of N6-methyladenosine (m⁶A) RNA methylation in the subject, thereby stimulating an immune response in the subject.

Embodiment 18 is the method of Embodiment 17, wherein the agent is an inhibitor of at least one of Methyltransferase-Like Protein 3 (METTL3), METTL14, and Wilms Tumor 1 Associated Protein (WTAP).

Embodiment 19 is the method of Embodiment 17 or 18, wherein the agent is a nucleic acid, a small molecule, or a polypeptide, such as an antibody or an antigen binding fragment thereof.

Embodiment 20 is the method of any of Embodiments 17 to 19, wherein the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6.

Embodiment 21 is the method of Embodiment 20, wherein the polynucleotide sequence is (GGACU)n, wherein n is an integer of 2 to 6.

Embodiment 22 is the method of Embodiment 21, wherein the oligonucleotide consists of the polynucleotide sequence of 5′ GGACUGGACUGGACUGGACU 3′ (SEQ ID NO: 1), which optionally contains one or more nucleoside modifications.

Embodiment 23 is the method of any one of Embodiments 20 to 22, wherein one or more nucleoside in the oligonucleotide is modified.

Embodiment 24 is the method of Embodiment 23, wherein one or more nucleoside in the oligonucleotide has a 2′-fluoro modification, preferably, one or more adenosine residues in the oligonucleotide has a 2′-fluoro modification.

Embodiment 25 is the method of any of Embodiments 22 to 24, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 26 is the method of any of Embodiments 20 to 25, wherein the oligonucleotide is conjugated via a linker to a targeting moiety.

Embodiment 27 is the method of any of Embodiments 20 to 26, wherein the oligonucleotide is administered intravenously or subcutaneously.

Embodiment 28 is the method of any of Embodiments 18 to 27, wherein the subject is administered with the effective amount of the agent in combination with a drug, preferably an anti-viral drug, an anti-inflammatory drug, or an immune modulator.

Embodiment 29 is an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 30 is the oligonucleotide of Embodiment 29, wherein the oligonucleotide is conjugated via a linker to a targeting moiety.

Embodiment 31 is a pharmaceutical composition comprising the oligonucleotide of Embodiment 29 or 30 and a pharmaceutically acceptable carrier.

Embodiment 32 is a pharmaceutical composition comprising a drug, such as an anti-viral active agent, preferably an anti-influenza drug, more preferably a neuraminidase inhibitor, such as Zanamivir or Oseltamivir, and an agent that decreases the level of N6-methyladenosine (m⁶A) RNA methylation in a subject, preferably the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6, more preferably the agent is a RNA oligonucleotide consisting of the polynucleotide sequence of 5′ GGACUGGACUGGACUGGACU 3′ (SEQ ID NO: 1), which optionally contains one or more nucleotide modifications, and most preferably the agent is an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 33 is a method of treating a disease in a subject in need thereof, comprising administering to the subject the pharmaceutical composition of Embodiment 31 or 32, wherein the disease is a disease in which stimulation of an immune response would be beneficial.

Embodiment 34 is a kit comprising a drug, such as an anti-viral active agent, preferably an anti-influenza drug, and an agent that decreases the level of N6-methyladenosine (m⁶A) RNA methylation in a subject, preferably the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6, more preferably the agent is a RNA oligonucleotide consisting of the polynucleotide sequence of 5′ GGACUGGACUGGACUGGACU 3′ (SEQ ID NO: 1), which optionally contains one or more nucleotide modifications, and most preferably the agent is an RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 35 is the kit of Embodiment 34, wherein the oligonucleotide and the anti-viral active agent are present in the same composition.

Embodiment 36 is the kit of Embodiment 34, wherein the oligonucleotide and the anti-viral active agent are present in different compositions.

Embodiment 37 is the kit of any of Embodiments 34 to 36, wherein the anti-viral active agent is a neuraminidase inhibitor.

Embodiment 38 is the kit of Embodiment 37, wherein the neuraminidase inhibitor is Zanamivir or Oseltamivir.

Embodiment 39 is a method of preventing or treating drug resistance in a subject administered a drug, the method comprising administering an effective amount of a nucleic acid inhibitor to the subject.

Embodiment 40 is the method of Embodiment 39, wherein the nucleic acid inhibitor is an inhibitor of at least one of Methyltransferase-Like Protein 3 (METTL3), Methyltransferase-Like Protein 14 (METTL14), and Wilms Tumor 1 Associated Protein (WTAP).

Embodiment 41 is the method of Embodiment 39 or 40, wherein the nucleic acid inhibitor is an oligonucleotide.

Embodiment 42 is the method of Embodiment 41, wherein the oligonucleotide is an RNA oligonucleotide.

Embodiment 43 is the method of Embodiment 41 or 42, wherein the oligonucleotide is a steric blocker.

Embodiment 44 is the method of any or Embodiments 41 to 43, wherein the oligonucleotide comprises the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6.

Embodiment 45 is the method of Embodiment 44, wherein the oligonucleotide consists of the polynucleotide sequence of 5′ GGACUGGACUGGACUGGACU 3′ (SEQ ID NO: 1), which optionally contains one or more nucleoside modifications.

Embodiment 46 is the method of Embodiment 44 or 45, wherein one or more nucleosides in the oligonucleotide is modified.

Embodiment 47 is the method of Embodiment 45 or 46, wherein one or more nucleoside in the oligonucleotide has a 2′-fluoro modification, preferably, one or more adenosine residues in the oligonucleotide has a 2′-fluoro modification.

Embodiment 48 is the method of any of Embodiments 45 to 47, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

Embodiment 49 is the method of any of Embodiments 39 to 48, wherein the drug is an anti-viral drug.

Embodiment 50 is the method of Embodiment 49, wherein the anti-viral drug is an anti-influenza drug.

Embodiment 51 is the method of Embodiment 50, wherein the anti-influenza drug is a neuraminidase inhibitor.

Embodiment 52 is the method of Embodiment 51, wherein the neuraminidase inhibitor is Zanamivir or Oseltamivir.

Embodiment 53 is a method of stimulating an immune response in a subject in need thereof, the method comprising administering to the subject an effective amount of a nucleic acid inhibitor.

Embodiment 54 is the method of Embodiment 53, wherein the nucleic acid inhibitor is an inhibitor of at least one of Methyltransferase-Like Protein 3 (METTL3), Methyltransferase-Like Protein 14 (METTL14), and Wilms Tumor 1 Associated Protein (WTAP).

Embodiment 55 is the method of Embodiment 53 or 54, wherein the nucleic acid inhibitor is an oligonucleotide.

Embodiment 56 is the method of Embodiment 55, wherein the oligonucleotide is an RNA oligonucleotide.

Embodiment 57 is the method of Embodiment 55 or 56, wherein the oligonucleotide is a steric blocker.

Embodiment 58 is the method of any of Embodiments 55 to 57, wherein the oligonucleotide comprises the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6.

Embodiment 59 is the method of Embodiment 58, wherein the oligonucleotide consists of the polynucleotide sequence of 5′ GGACUGGACUGGACUGGACU 3′ (SEQ ID NO: 1), which optionally contains one or more nucleoside modifications.

Embodiment 60 is the method of Embodiment 58 or 59, wherein one or more nucleosides in the oligonucleotide is modified.

Embodiment 61 is the method of Embodiment 59 or 60, wherein one or more nucleoside in the oligonucleotide has a 2′-fluoro modification, preferably, one or more adenosine residues in the oligonucleotide has a 2′-fluoro modification.

Embodiment 62 is the method of any of Embodiments 59 to 61, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

### EXAMPLES

The following examples of the present disclosure are to further illustrate the nature of the present disclosure. It should be understood that the following examples do not limit the disclosure and that the scope of the present disclosure is to be determined by the appended claims.

The experimental methods used in the following examples, unless otherwise indicated, are all ordinary methods. The reagents used in the following embodiments, unless otherwise indicated, are all purchased from ordinary reagent suppliers.

### Example 1

### Materials and Methods

### Cellular RNA, Viral RNA Extraction and m6A Quantification

Viruses were collected from MDCK supernatant 7 days post infection, spun at 2500 rpm for 10 mins, and RNA was extracted using a Qiagen viral RNA extraction kit (Cat #52904, Qiagen). RNA concentration was measured using a nanodrop (Thermo Fisher). Total m⁶A levels were measured using 2000 ng of total RNA m⁶A quantification kit (Cat #AB185912, Abcam). To extract cellular RNA, supernatant was removed, cells were washed once with ice cold PBS, and RNA was extracted using an RNA extraction kit (Cat #74104, Qiagen) according to the manufacturer's instructions.

### EC50 Determination

EC50 was determined using a 96 well format. The top concentration (dependent on EC50 for each drug) was serially diluted ⅓ dilution in a deep well block. After the addition of drugs, cells were incubated for 24 hrs followed by the addition of virus. Virus was incubated with cells for either 72, or 120 hrs, and cell viability was determined using cell titer glow (Cat #G7570, Promega) or viral levels were determined using a munana assay (Cat #4457091, Thermo Fisher) according to the manufacturer's protocol.

### Transfections

MDCK cells were transfected using lipofectamine 2000. Briefly, 100 nM of oligonucleotides were transfected per well (Control RNA: 5′ GGGCUGGGCUGGGCUGGGCU 3′ (SEQ ID NO: 3); Target RNA: 5′ GGACUGGACUGGACUGGACU 3′ (SEQ ID NO: 1); Stable RNA: 5′ GG/i2FA/CUGG/i2FA/CUGG/i2FA/CUGG/i2FA/CU 3′ (SEQ ID NO: 2)) using Lipofectamine 2000 (Cat #11668010, Thermo Fisher), and after a 24 hr incubation the medium was replaced with DMEM containing 10% FBS. The oligonucleotides were synthesized by IDT.

### Immunoprecipitation Assays and Western Blotting

Immunoprecipitations were done using 5′-biotin labeled oligos. Briefly, 20×10\hat{ }6 of 293T cells were lysed using NP40 lysis buffer (Cat #FNN0021, Thermo Fisher) supplemented with protease inhibitors (Cat #78438, Thermo Fisher), the lysate was spun at 4° C. for 10 min, and supernatant was isolated. Total protein concentration was measured using a BCA assay (Cat #23227, Thermo Fisher) according to the manufacturer's protocol. 100 μg of total lysate was incubated with 100 nM biotinylated oligo supplemented with protease and RNAse inhibitors (Cat #N8080119, Thermo Fisher) at 4° C. for 1 hr on a rocker, and then 304, of streptavidin coated beads (Cat #11206D, Thermo Fisher) were added and incubated for an additional 2 hrs. Beads were washed 3× with ice cold NP40 lysis buffer, and beads were boiled with SDS sample buffer for 10 mins at 100° C.

### Example 2

### Viral RNA Methylation Increases with Zanamivir-Resistant Influenza A Virus

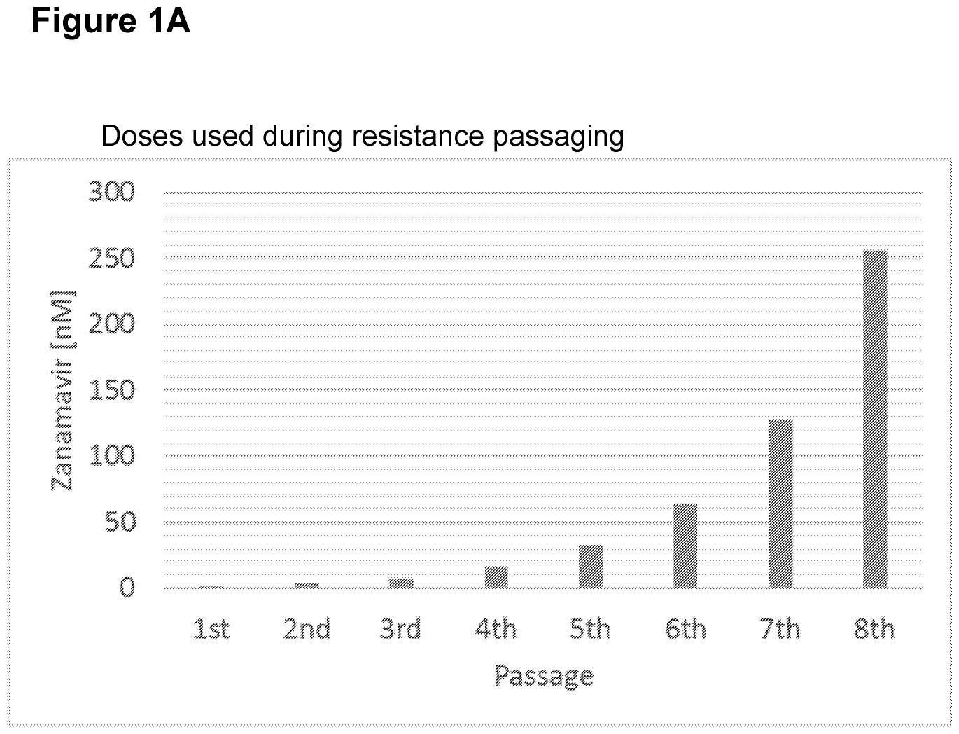

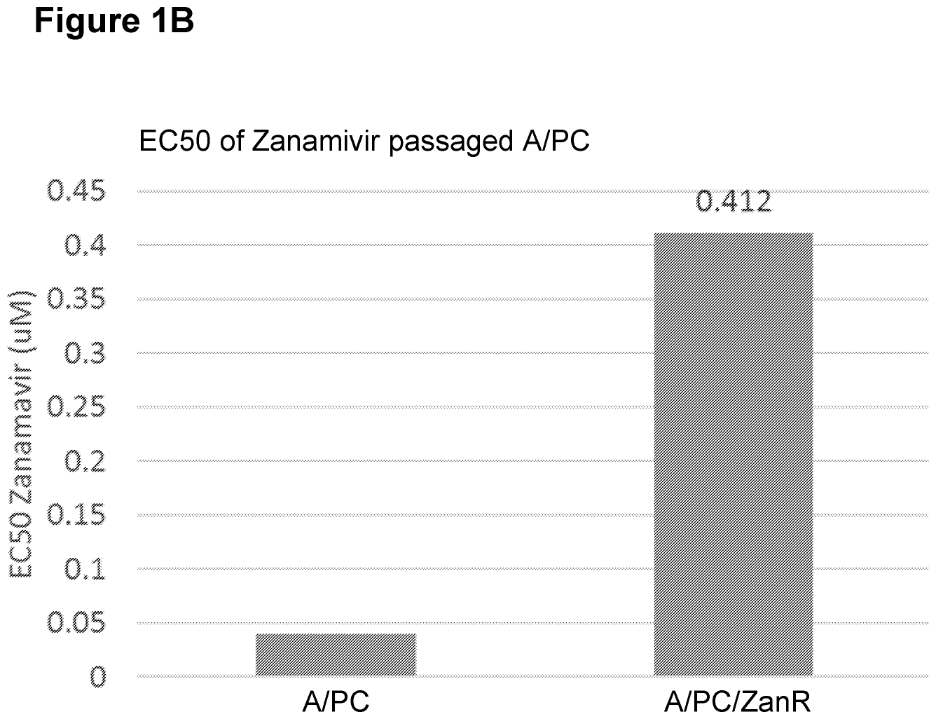

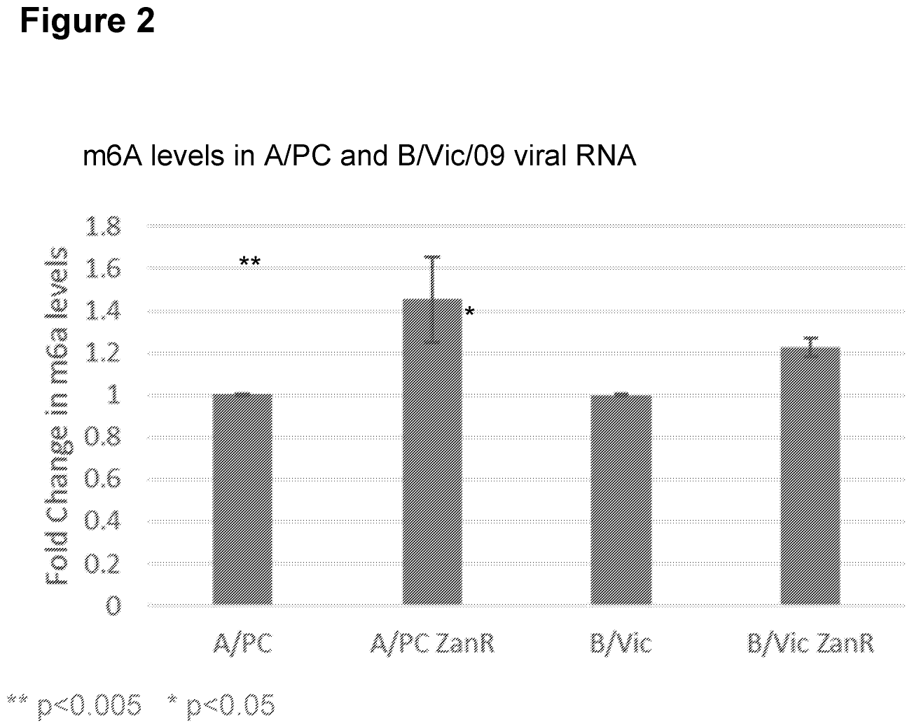

To generate drug resistant influenza A virus, IVA was serially passaged in MDCK cells. 5×10\hat{ }6 MDCK (Cat #CC134, ATCC) cells were seeded in 6-well plates. The cells were infected with IVA/PC virus at an MOI and Zanamivir (Cat #SML0492, Sigma), starting at the IC50 of 2 nM. The cells were visually inspected, and after significant cytopathic effect was observed, the next passage was performed with increasing doses of Zanamivir. The dose was increased 2-fold every passage until approximately 415 nM (FIG. 1). To evaluate the possible changes in m⁶A methylation in wild type and drug resistant IVA and IVB virus, viral RNA was extracted and m⁶A was measured in an m⁶A-specific ELISA, as described above. In each virus, a significant increase in total m⁶A was detected (FIG. 2), suggesting a role for m⁶A in acquired drug resistance.

### Example 3

### Modulators of RNA Methylation Alter Drug Resistance

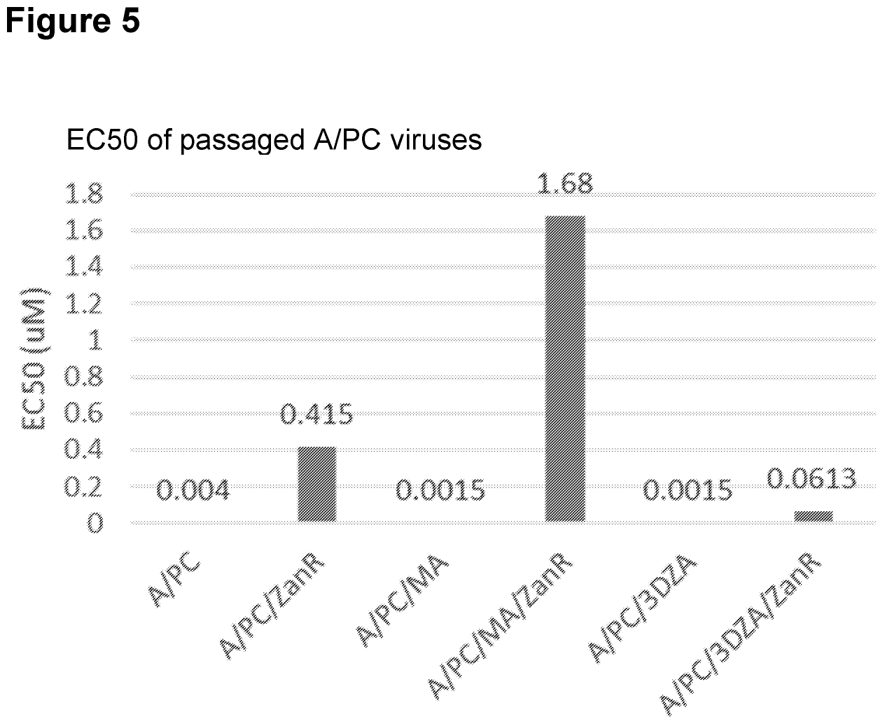

It has been demonstrated that altering RNA methylation using 3-deaza adenosine (3DZA) inhibited the generation of drug resistant IVA (Scholtissek and Müller, Archives of Virology, 1991). Based on this information, the role of RNA modulators in regulating drug resistance was tested. 3DZA (25 uM) was used to inhibit total cellular m⁶A, and MA (50 uM) was used to increase total cellular m⁶A (Fustin et al., Cell, 2013, Huang et al., Nucleic Acids Research, 2014). As shown in FIG. 5, increasing m⁶A RNA methylation levels by treatment with MA potentiates viral resistance to Zanamivir.

Next, resistant passaging with Zanamivir in the presence of either 3DZA or MA was performed. 5×10\hat{ }6 MDCK (Cat #CC134, ATCC) cells were seeded in 6-well plates. The cells were treated with 50 μM 3DZA (Cat #D8296, Sigma) or 100 μM MA (Cat #M4531, Sigma). 24 hours later, media was replaced with MEM with 0.3% FBS 10% PS and 1:5000 TPCK trypsin (Cat #T1426, Sigma), and each well was infected with IVA/PC virus at various MOI and concentrations of Zanamivir (Cat #SML0492, Sigma). The cells were visually inspected, and after significant cytopathic effect was observed, the next passage was performed with increasing doses of Zanamivir. The levels of m6A were measured using m6A ELISA. As shown in FIG. 5, an increase in IC50 was observed with MA (1.68 uM) and a decrease in IC50 was observed with 3DZA (0.0618 uM).

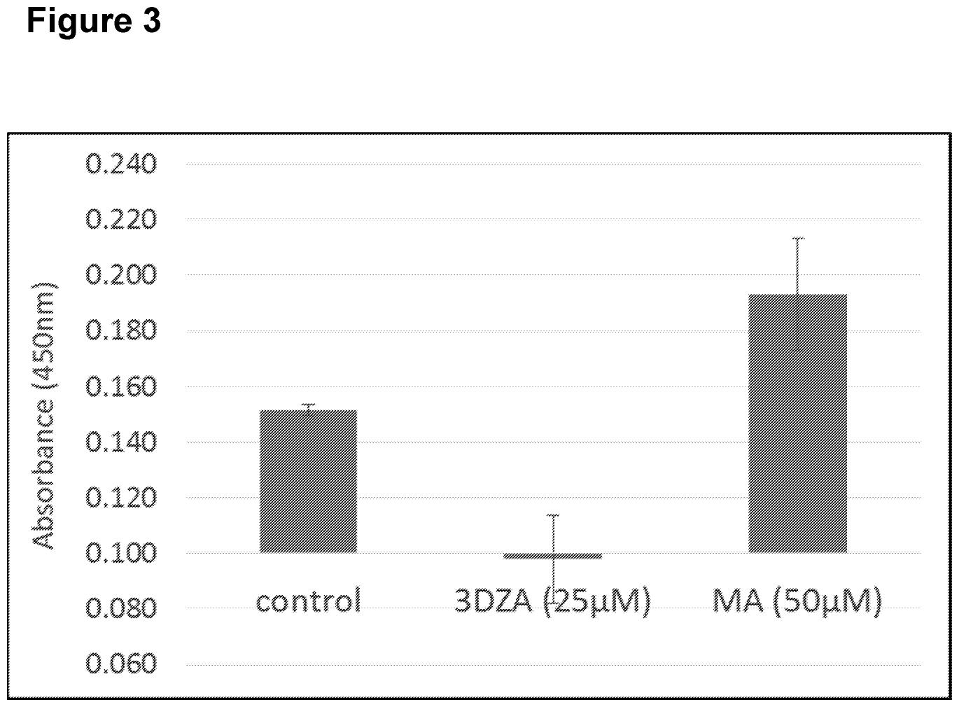

Total m⁶A levels in viral RNA were measured after treating MDCK cells with 3DZA and MA. As shown in FIG. 3, an increase of m⁶A in MA-treated cells and a reduction of m⁶A in 3DZA-treated cells was observed, suggesting that targeting host methytransferases also alters m⁶A levels in viral RNA. Zanamivir-resistant influenza A viruses have been shown to harbor mutations that directly cause resistance.

### Example 4

### Targeting METTL3 with Oligonucleotide Steric Blockers

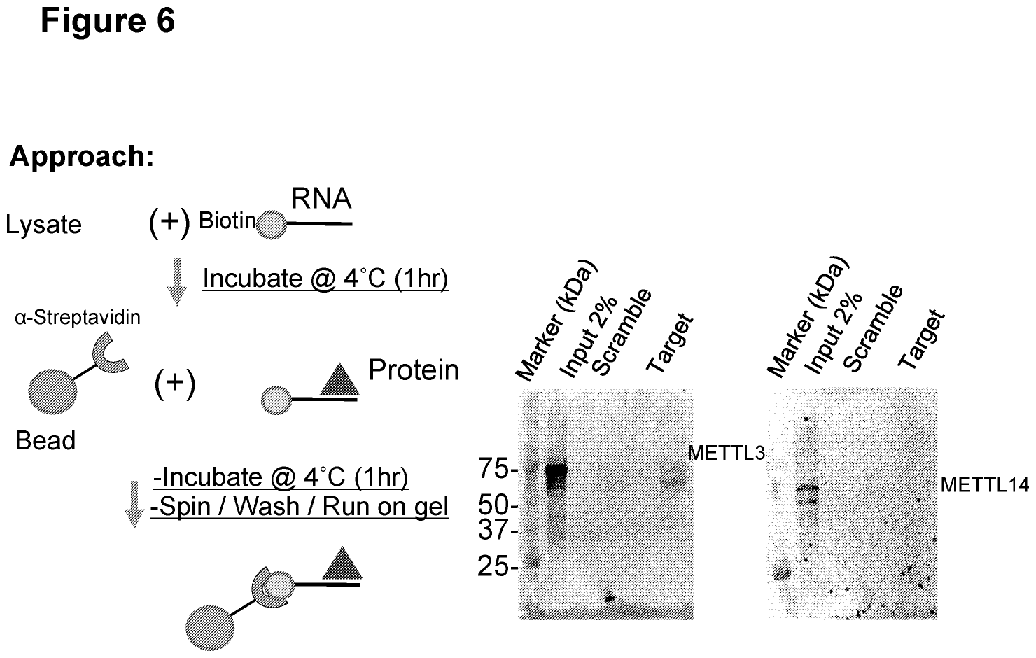

Since a change in drug resistance using RNA methylation modulators MA and 3DZA was observed, an oligonucleotide inhibitor composed of 4 repeats of GGACU that could competitively inhibit METTL3 activity was designed (FIG. 6).

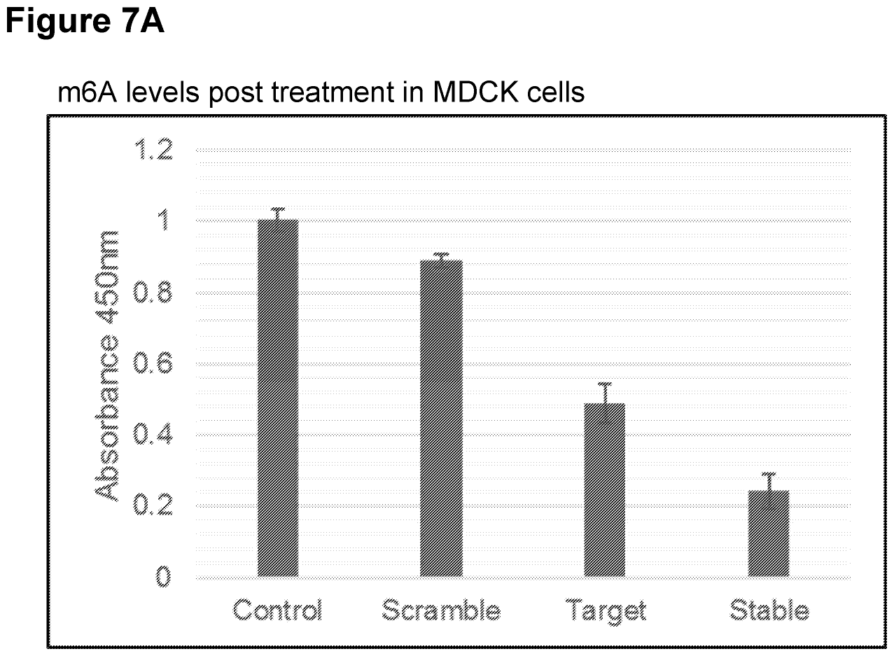

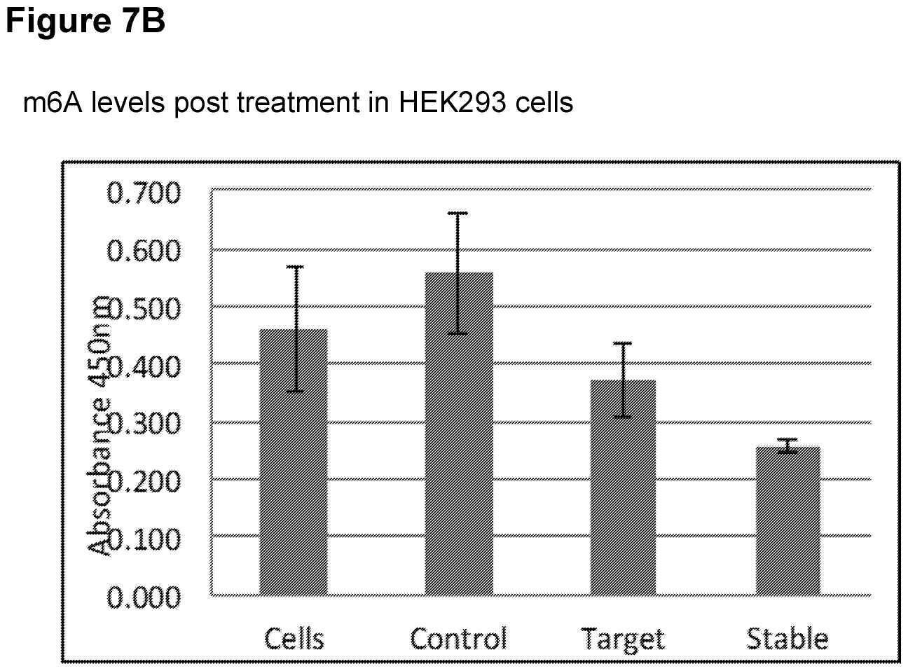

To determine inhibition of m⁶A, MDCK and 293 cells were transfected with 100 nM of either the control, target or stable oligo. Levels of m⁶A were determined 72 hrs after the transfections. An 80% or 50% inhibition of m⁶A levels was observed in MDCK and 293 cells transfected with the stable oligo, respectively (FIG. 7).

It was tested whether the target sequence bound METTL3 using an RNA pulldown assay with 3′-biotin labeled oligo incubated in 293 total cell lysate. As shown in FIG. 6, RNA oligonucleotides according to embodiments of the present disclosure can bound METTL3, but not METTL14, in vivo.

### Example 5

### Targeting Host METTL3 with Oligonucleotide Steric Blockers Inhibits Zanamivir Resistance

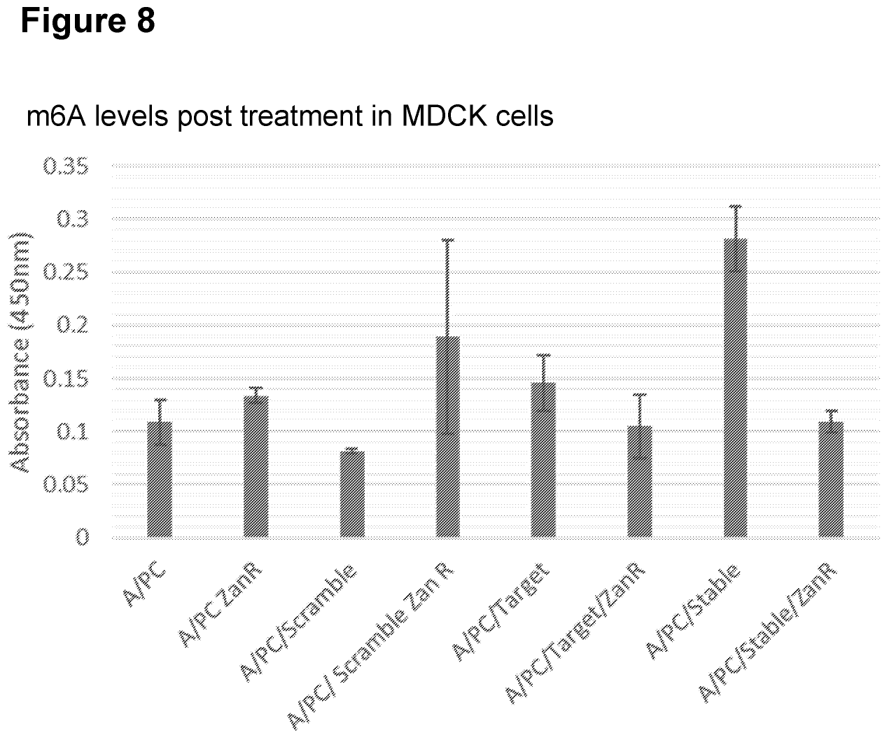

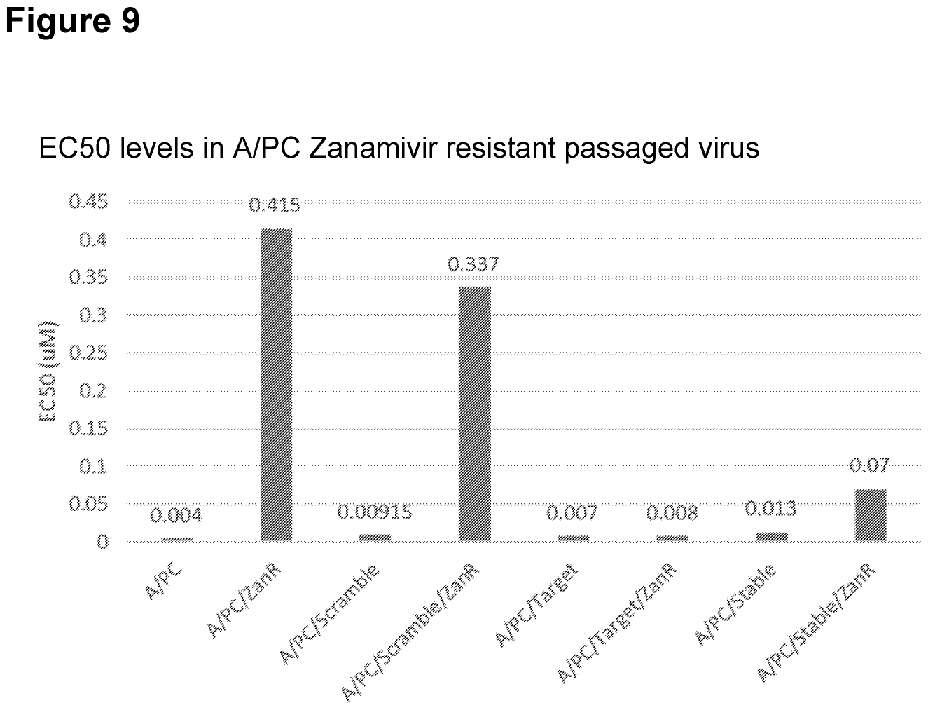

Efficient binding of METTL3 and inhibition of host m⁶A suggested that RNA oligonucleotides according to embodiments of the present disclosure could serve as steric blockers of METTL3 and could be used as adjuvants with anti-viral active agents such as Zanamivir to inhibit drug resistance. To test this, MDCK cells were treated with target or stable oligos (FIG. 9) alone or in combination with Zanamivir, and the EC50 levels were determined after 10 rounds of selection. As shown in FIG. 9, a reduction in drug resistance with cells pre-treated with either target or stable oligo compared to control (Scramble: 337 nM versus Target: 8 nM and stable: 70 nM) was observed. Furthermore, a concomitant decrease in m⁶A methylation in corresponding viral RNA was observed (FIG. 8), suggesting that the changes are due to changes in viral RNA m⁶A levels.

While the invention has been described in detail, and with reference to specific embodiments thereof, it will be apparent to one of ordinary skill in the art that various changes and modifications can be made therein without departing from the spirit and scope of the present disclosure.

1. A method of reducing drug resistance in a subject in need thereof, the method comprising administering to the subject an effective amount of an agent, preferably an oligonucleotide, that decreases the level of N6-methyladenosine (m⁶A) RNA methylation in the subject, thereby reducing drug resistance in the subject.

2. The method of claim 1, wherein the agent, preferably the oligonucleotide, is an inhibitor of at least one of Methyltransferase-Like Protein 3 (METTL3), METTL14, and Wilms Tumor 1 Associated Protein (WTAP).

3. The method of claim 1, wherein the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6.

4. The method of claim 3, wherein the RNA oligonucleotide consists of the polynucleotide sequence of 5′ GGACUGGACUGGACUGGACU 3′ (SEQ ID NO: 1).

5. The method of claim 4, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

6. The method of claim 1, wherein the subject is administered with the effective amount of the agent in combination with an anti-influenza drug.

7. The method of claim 6, wherein the anti-influenza drug is a neuraminidase inhibitor.

8. The method of claim 7, wherein the neuraminidase inhibitor is selected from the group consisting of Zanamivir and Oseltamivir.

9. A method of stimulating an immune response in a subject in need thereof, the method comprising administering to the subject an effective amount of an agent, preferably an oligonucleotide, that decreases the level of N6-methyladenosine (m⁶A) RNA methylation in the subject, thereby stimulating an immune response in the subject.

10. The method of claim 9, wherein the agent, preferably the oligonucleotide, is an inhibitor of at least one of Methyltransferase-Like Protein 3 (METTL3), METTL14, and Wilms Tumor 1 Associated Protein (WTAP).

11. The method of claim 9, wherein the agent is an RNA oligonucleotide comprising the polynucleotide sequence of (RRACH)n, wherein at least one R is independently guanosine and the other is independently guanosine or adenosine, A is adenosine that is either methylated or unmethylated, C is cytidine, H is independently adenosine, cytidine or uridine, and n is an integer of 2 to 6, optionally, one or more nucleoside in the oligonucleotide is modified.

12. The method of claim 11, wherein the RNA oligonucleotide consists of the polynucleotide sequence of 5′ GGACUGGACUGGACUGGACU 3′ (SEQ ID NO: 1), optionally, one or more nucleoside in the oligonucleotide is modified.

13. The method of claim 12, wherein the oligonucleotide consists of the polynucleotide sequence of 5′ GG/i2FA/CUGG/i2FA/CUGG/i2FA/CUGG/i2FA/CU 3′ (SEQ ID NO: 2), wherein i2FA represents 2′-fluoro-adenosine.

14. An RNA oligonucleotide consisting of 5′ GG/i2FA/CUGG/i2FA/CUGG/i2FA/CUGG/i2FA/CU 3′ (SEQ ID NO: 2), wherein i2FA represents 2′-fluoro-adenosine.

15. A pharmaceutical composition comprising the oligonucleotide of claim 14 and a pharmaceutically acceptable carrier.

16. The pharmaceutical composition of claim 15, further comprising an anti-viral active agent, preferably an anti-influenza drug.

17. A method of treating a disease in a subject in need thereof, comprising administering to the subject the pharmaceutical composition of claim 15, wherein the disease is a disease in which stimulation of an immune response would be beneficial.

18. (canceled)

19. (canceled)

20. (canceled)

21. A method of preventing or treating drug resistance in a subject administered a drug, the method comprising administering to the subject an effective amount of an agent, preferably an oligonucleotide, that decreases the level of N6-methyladenosine (m⁶A) RNA methylation in the subject.

22. The method of claim 21, wherein the agent, preferably the oligonucleotide, is an inhibitor of at least one of Methyltransferase-Like Protein 3 (METTL3), Methyltransferase-Like Protein 14 (METTL14), and Wilms Tumor 1 Associated Protein (WTAP).

23. The method of claim 21, wherein the agent is an oligonucleotide.

24. The method of claim 23, wherein the oligonucleotide is an RNA oligonucleotide.

25-40. (canceled)


SEQUENCE LISTING (3 sequences)

SEQ ID NO: 1 - RNA, 20 nt, Artificial Sequence
  synthetic oligonucleotide
ggacuggacuggacuggacu

SEQ ID NO: 2 - RNA, 20 nt, Artificial Sequence
  synthetic oligonucleotide
  i2FA (3)..(3)
  i2FA (8)..(8)
  i2FA (13)..(13)
  i2FA (18)..(18)
ggacuggacuggacuggacu

SEQ ID NO: 3 - RNA, 20 nt, Artificial Sequence
  synthetic oligonucleotide
gggcugggcugggcugggcu